(19) United States
(12) Patent Application Publication
(10) Pub. No.: US 2009/0209831 A1
(43) Pub. Date: Aug. 20, 2009

(54) Imaging methods for visualizing implanted living cells

(75) Inventors: John Kucharczyk, Reno, NV (US); Mark A. Litman, Edina, MN (US)

Correspondence Address: Mark A. Litman & Associates, P.A., York Business Center, Suite 205, 3209 West 76th Street, Edina, MN, 55435, US

(73) Assignee: NexGen Medical Systems, Incorporated

(21) Appl. No.: 12/321,139

(22) Filed: Jan. 15, 2009

Related U.S. Application Data
(63) Continuation-in-part of application No. 09/606,137, filed on Jun. 28, 2000.
(60) Provisional application No. 61/011,130, filed on Jan. 15, 2008.

(51) Int. Cl. A61B 5/00; A61B 5/055

(52) U.S. Cl. 600/301; 600/410

(57) ABSTRACT

A method is provided herein for indicating viability of cells grown on a rejection inert tissue cytoarchitecture or scaffolding with a medical device that supports at least one sensing function comprising:
non-destructively observing a region of a patient to where cells have been transplanted;
guiding the medical device to said region of a patient using the non-destructive observation;
positioning said medical device within said region of a patient using the non-destructive observation to assist in the positioning;
sensing a property within said region of a patient that is indicative of cell viability or nonviability; and
using data from sensing said property within said region to indicate cell viability from a transplant with the region. Magnetic Resonance Imaging is a particularly useful format for non-destructive observation of the region.

### RELATED APPLICATIONS DATA

This application is a Continuation-in-part Application of Pending U.S. patent application Ser. No. 09/606,137, filed Jun. 28, 2000 and claims priority from U.S. Provisional Patent Application Ser. No. 61/011,130, filed 15 Jan. 2008.

### BACKGROUND OF THE INVENTION

1. Field of the Invention

This invention relates to non-invasive imaging methods for visualizing implanted living cells and for assessing the metabolic and physiologic viability of implanted living cells and their progeny.

2. Background of the Art

Current technologies make it possible to measure intracranial pressure (U.S. Pat. No. 5,107,847), deliver drugs in a rate-controlled manner (U.S. Pat. No. 5,836,935), infuse various substances into the brain (U.S. Pat. No. 5,720,720), and convey fluids out of the brain (U.S. Pat. No. 5,772,625). Further illustrative examples of functional intracranial probes include U.S. Pat. No. 5,843,150 to Dreessen et al., U.S. Pat. No. 5,861,019 to Sun et al., U.S. Pat. No. 5,843,148 to Gijsbers et al., U.S. Pat. No. 5,820,589 to Torgerson et al., U.S. Pat. No. 5,821,011 to Taylor et al., U.S. Pat. No. 5,826,576 to West, U.S. Pat. No. 5,858,009 to Jonkman, and PCT application WO9807367A1 to Jolecz et al.

U.S. Pat. Nos. 5,125,888, 5,707,335, 5,779,694, and 5,843,093 disclose intracranial probes that can be positioned within the brain by magnetic stereotaxis, which probes are also visible under magnetic resonance (MR) imaging. Several types of implantable neurostimulator devices, such as those described in U.S. Pat. No. 5,344,439 to Otten, U.S. Pat. No. 4,800,898 to Hess et al., and U.S. Pat. No. 4,549,556 to Tarjan et al., have also been disclosed.

U.S. Pat. Nos. 5,711,316, 5,713,923, 5,735,814, 5,832,932, and 5,978,702 disclose implantable pumps and catheters for infusing drugs into the brain to treat movement disorders, wherein a sensor detects the symptoms resulting from the movement disorder and a microprocessor algorithm analyzes the output from the sensor to regulate the amount of drug delivered to the brain. U.S. Pat. No. 5,607,418 to Arzbaecher discloses an implantable drug delivery apparatus comprising a housing with a plurality of drug compartments that can be opened in a timed manner by, for example, a gas-generating element to release the drugs into the tissue.

Each of the above cited patents offers advantages for providing and/or monitoring physiologic parameters related to drug therapy.

Parkinson's disease is characterized by a deficiency of the neurotransmitter dopamine within the striatum of the brain, secondary to damage or destruction of the dopamine secreting cells of the substantia nigra in the midbrain. To date, direct intraparenchymal delivery of purified or synthetic dopamine, or its precursors, analogs or inhibitors has not demonstrated clear therapeutic benefit because of various problems associated with drug delivery, stability, dosage and cytotoxicity. In other neurodegenerative disease states, biologically active macromolecules appear to provide benefits by ameliorating the disease process or stimulating responses that result in therapeutic improvement. For example, models of Alzheimer's disease have been shown to benefit from the introduction of protein growth factors in vivo. Models of primary brain tumors have demonstrated therapeutic responses by introducing cytokines designed to stimulate the immune response against the tumor cells. However, similar difficulties with reliable continuous delivery of these agents in actual clinical settings have yet to be resolved.

Implantable miniature osmotic pumps have been used to provide a continuous supply of drugs or other active biologic factors to the brain and other tissues at a controlled rate. Reservoir limitations as well as drug solubility and stability have, however, restricted the usefulness of this technology. Controlled sustained release of dopamine for the treatment of Parkinson's disease has been attempted from within bioabsorbable microcapsules, such as disclosed by U.S. Pat. No. 4,883,666 to Sabel, et al. However, this method appears to rely on surface erosion of the bioabsorbable polymer, which is in turn influenced by various hydrolytic events, thereby increasing the likelihood of drug degradation, and rendering predictable release rates difficult. A further problem appears to be attributable to limited surface area for diffusion per unit volume of larger size microspheres, such that only a limited volume of cells can be loaded into a single microcapsule.

Macroencapsulation, which generally involves loading cells into hollow fibers and then sealing the ends of the fibers, has also been used to deliver therapeutic drugs into the central nervous system. Exemplary of the macroencapsulation approach to drug delivery is U.S. Pat. No. 4,892,538 to Aebischer, et al., which discloses methods for delivery or a neurotransmitter to a target tissue from an implanted, neurotransmitter-secreting cell culture within a semi-permeable membrane, wherein the surgically implanted cell culture device may be retrieved from the brain, replaced or recharged with new cell cultures, and re-implanted. U.S. Pat. No. 5,106,627 to Aebischer et al. additionally discloses a method for the combined delivery of neurotransmitters and growth factors from implanted cells encapsulated within a semi-permeable membrane. However, while these methods may offer the advantage of easy retrievability, the encapsulation of cells within macrocapsules implanted in the brain is often complicated by unreliable closure of the reservoir resulting in inconsistent results.

Studies utilizing implantation of cells capable of producing and secreting neuroactive factors directly into brain tissue have demonstrated that Parkinson's disease symptoms can be improved by transplanting fetal dopamine cells into the putamen of the brain of patients with Parkinson's disease. U.S. Pat. No. 5,487,739 to Aebischer, et al. discloses a cell therapy delivery method utilizing a cannula, obturator, and implantable cells, wherein the biologically active factors diffuse into brain tissue through an implanted semi-permeable membrane. U.S. Pat. No. 5,006,122 to Wyatt, et al. discloses an apparatus for transplanting tissue into a brain, comprising a stereotactic device for inserting a guide cannula to a target location within the brain into which a second cannula containing the tissue transplant is inserted and the tissue is deposited. Many factors have been shown to influence long-term graft viability, including the age of the donor tissue, the site of the graft placement, the specificity of donor tissue, and the techniques used in the preparation of the tissue to be transplanted. However, even when all of the above factors are taken into account, only 3-20% of the implanted cells survive more than seven days (see for example article by A. Bjorkland, “Better cells for brain repair” Nature 1993; 363; 414-415).

One potential factor, which may account for the high attrition rate of transplanted cells is a lack of nutritive support. Angiogenesis in cell grafts has been shown to occur only after approximately three days post-implantation, which suggests that most implanted cells may die within the first 48 to 72 hours after implantation when they are presumably totally dependent on local diffusion for nutritional support (see for example article by Watts et al., “The development of intracerebral cell-suspension implants is influenced by the grafting medium.” Cell Transplantation 1998; 7; 573-583). The precise mechanisms of survival of implanted cells until a vascular supply becomes established is not known, although it may be influenced by the oxygen tension of the local environment during the course of graft vascularization (see for example article by Stokes et al., “Oxygen transport in intraspinal grafts: Graft-host relations.” Experimental Neurology 1991; 111; 312-323).

Another major problem for cell implant therapy is the limited and variable supply of human fetal tissue. This has limited research and clinical neural transplantation programs to very small number of patients. Fetal pig neural cells have also been shown to survive in an immuno-suppressed parkinsonian patient. Improvements in the quality of transplantation also appear to be emerging, as exemplified in recent studies. For example, Zawada, et al., Nature Medicine, Vol. 4, pps. 568-574 (1998) have demonstrated that somatic cell cloning can efficiently produce transgenic animal tissue for treating parkinsonism.

An alternative approach is to isolate and expand neural precursor cells from the human central nervous system. It is now possible to surgically remove neural progenitor cells from a patient, grow the cells in culture, insert therapeutic genes, and then replace the transfected cells back into the patient's brain. Copending U.S. Patent Application Ser. No. 60/177,263 by Kucharczyk, Gillies, Broaddus, and Fillmore titled “Device and Method for Image Guided Cell Therapy” discloses an improved image-guided device and method to deliver cells that can produce biologically active factors to a target region of the brain. However, the ability to monitor ongoing cell viability non-invasively with MR imaging is not currently available.

Thus, there exists a need for a method to monitor non-invasively the ongoing viability of the cell implant, in particular to determine whether the cells are adequately perfused by the local microvasculature and continue to provide sustained delivery of the deficient biologically active factor. U.S. Pat. No. 5,190,744 to Rocklage et al. discloses MRI methods for evaluating local and regional tissue perfusion based on first pass tracking of a bolus of MRI contrast agent. U.S. Pat. No. 5,494,655 to Rocklage et al. additionally discloses MRI methods for evaluating local and regional tissue perfusion changes induced by administration of a vasodilatory or vasoconstrictive drug agent. U.S. Pat. No. 5,833,947 to Rocklage et al. further discloses MRI methods for evaluating local and regional tissue perfusion changes induced by surgical procedures.

Unlike the present invention, however, none of these patents disclose a method for quantitating the functional capillary density in the anatomic region of the cell implant, or for quantitatively determining the metabolic status of a population of living cells implanted into a tissue in a human body.

U.S. Pat. No. 6,026,316 (Kucharczyk and Moseley) and U.S. Pat. No. 6,061,587 (Kucharczyk and Moseley) describe methods and apparatus for the MR-assisted visualization of molecular level changes in composition from materials added or which have their in vivo production stimulated locally. Real time MR imaging at higher Tesla levels can provide images identifying concentration changes of these introduced and production-stimulated materials, particularly by using RF microcoils in the region where the materials are delivered or stimulated.

### SUMMARY OF THE INVENTION

No disclosures are known to be available that provide a method or device for image-guided, targeted delivery of cells, with and/or without supportive intracranial drug therapy, as well as a means for non-invasively monitoring the physiologic and metabolic status of the cell implant.

The present invention is particularly well suited for evaluating the efficacy of cell therapy of Parkinson's disease and other neurodegenerative diseases and disorders.

The present invention provides an MR imaging method for the local and controlled delivery into tissues of living cells and cell suspensions that can produce a biologically active factor, while reducing present levels of trauma by some corrective therapies or without causing trauma. The invention additionally discloses an imaging system to monitor the metabolic status of the transplanted cells and their assimilation into their tissue transplant environment based on MR indicators of cell growth and proliferation. According to the invention, metabolic changes arising from cell replication or cell death can be measured using MR imaging to detect changes in the field of therapy that have been induced by the success or failure of the procedure, for example, by identifying localized proton lactate and metabolite signals. The resulting MR data may then be used to provide a non-invasive assessment of cell viability. In one embodiment, the metabolic changes are measured by Magnetic Resonance by non-invasive in vivo proton-spectroscopy with local or volume RF-coils. In a embodiment, proton observable metabolites GABA, PCr, creatine, choline, and lactate are measured. Concentrations of lactate above about 2-6 millimolar may be used, for example, to indicate a significant occurrence of dying or dead cells.

According to the invention, viable cell implants can be distinguished from dead or dying cells based on quantitative regional indications of changes in signals resulting from the activity (or change in activity) of implanted cells, the inactivity of implanted cells, increased (or decreased) cell product due to increased (or decreased) numbers of active cells, and/or lactate to metabolite levels. In another embodiment of the present invention, a quantitative assessment of the populations of living to dead cells is obtained from the molar ratios of the proton signals that can be quantitated to indicate resulting changes in local chemistry environments, as from changes due from concentration changes in specific local chemistries resulting from converting lactate to metabolites. In a particularly preferred embodiment of the invention, local glucose turnover is monitored by non-invasive imaging technologies that can track, in vivo, C-13 labeled glucose introduced directly into brain tissues together with the cell implant. Glucose metabolism in the cell implant is assessed by observing the resulting data from in vivo conversion of the C-13 labeled glucose into C-13 labeled metabolic by-products. According to the invention, the levels and turnover rates of glucose utilization, as measured by the concentrations of the converted compounds, reflect the ongoing viability of the cell implant.

The in vivo conversion of the C-13 labeled glucose into C-13 labeled metabolic byproducts is directly observed by measuring the signal intensity at each of the chemicals shifts of the individual metabolic byproducts. That is, the shift or metabolic conversion of compounds can be directly monitored by measuring the amount of change between the different MR responsive shifts related to each of the respective original compound(s) and resultant compound(s). The metabolic molecules, each with a unique chemical shift, vary in signal strength as a result of the direct utilization of glucose, and they remain within the field of view of either the imaging system or within the field of view of the local RF coils. In this way, the C-13 labeled glucose signal will decrease while the signal intensities of the metabolic byproducts increase, the total amount of C-13 that was introduced by the labeled glucose remaining the same. Note that the magnetic resonance imaging or spectroscopy examples are sensitive to signal strengths as functions of chemical shift.

In a similar fashion, the analytical methods that may be use within the scope of the present invention may operate the same with an F-19 labeling process. The initial F-19 label signal intensity at the unique chemical shift of the F-19 metabolite is diminished as the metabolic byproducts containing now the metabolized F-19 nuclei appear in the MR system as different chemical shifts. In this fashion, a variety of nuclei may be labeled such as C-13, F-19, nitrogen-14, nitrogen-15, sulfur-33, deuterium, magnesium, manganese, iron or any magnetic resonance sensitive nucleus. The process of metabolizing the labeled nuclei leads to differing chemicals shifts depending on the metabolic byproducts. It is possible to identify distinct implantation results by providing cells with different markers either to distinct sites (where multiple therapies might be administered to different regions of the brain) or to the same site with different attendant treatments for the distinctly labeled cells (to assist in determining improved procedures or preferred original cell sources).

A software program may be ready established to oversee the analytical system or at least to supplement observations by the operator, technician, or doctor. For example, the software program may operate in a manner that includes at least some of the following steps:

Observe a Field of Proposed Cell Therapy

This can be done by measuring the response of that field to an imaging technology (e.g., MR, fluoroscopy, sonogram, etc.), and provide that information to a computational system, such as a PCU, microprocessor, circuit board, hardboard, mainframe, etc.)

Determine (either by direct observation or by computational analysis, either mentally or by computational device) components of the response that are associated with specific readable components (e.g., labels or natively responsive components, molecules or atoms (this is essentially developing a reading of the viewable area background or base-line)

Introducing Cells into the Field

Identifying at least some specific readable components introduced to the field by introduction of the cells

Comparing concentrations and/or the change in the range or region of presence (or the mere presence of) of readable components after cell introduction as compared to concentrations and/or the change in the range of presence (or mere presence of) of readable components prior to cell introduction by continued or periodic observation of the field

Observing Concentrations or Change in the Range of Presence of Readable Components

Qualitatively and/or quantitatively determining changes and/or rates of changes and/or locations of changes in the concentration and/or presence of the readable components.

Recent technological developments have been announced in the field of tissue and organ engineering, wherein viable new organs have been developed by adding cells to organ support structures (that is, underlying physical structure or cytoarchitecture on existing tissue of organs without functioning organ tissue, such as the skeletal support for a heart without muscles, without living functionality, e.g., a heart beat, hormone producing functionality, oxygen exchange functionality, nutrient absorption and utilization functionality, and the like) that act as a scaffold for the new cells. An artificial scaffold may also be inserted if it is cell rejection inert. It is a likely scenario in the production and growth of independent and complete organs by this technology that defects will occur in the growth of individual organs, and that these defects might appear as localized defects wherein certain sections of the organ will perform differently (including inert activity) than other sections of the organ. It is also likely, as with certain fertility treatments, that to assure or increase the potential for success, multiple organs will be grown at the same time and that only organs identified as the best results of the organ growth techniques will be selected for implantation. A need clearly exists for useful, non-invasive methods for identifying highest performing organs according to measurable parameters.

As noted above, the sophistication of the observation may be varied by including additional steps (beyond those listed above) or by removing some of the steps. A useful analysis of the cell survival even may be performed by a continuous single step process of continuously monitoring a field while qualitatively or quantitatively determining the presence and/or location of readable components. More sophisticated software and hardware systems could evaluate the mass flow vectors in the field, determine specific areas within the field with differing changes in rates or concentrations of metabolites, and therefore identifying subsections of cell survival within the general field, and comparing data for specific or undefined anomalies in data obtained. Such a partially or completely automated analytic system provides a highly useful and rapid response methodology for assisting in the immediate evaluation of levels of success of cell implantation. This would enable implantation surgery to be modified during an original procedure.

Although the time of duration of the initial procedure may be extended, the need for one or more subsequent procedures may be eliminated. The limitation on the time frame or time interval between measurements to provide an accurate gauge of the viability of the transplanted cells depends upon the environment and particulars of each implantation and the activity of the particular class of cells. For example, certain cells may be actively metabolizing materials, and a short period between data sets or a continuous sequence of measurements may indicate a rate or a trend. Other cells may metabolize very slowly and would require a longer time period before meaningful data could be collected and analyzed.

In another preferred embodiment of the invention, the viability of the cell implant is also assessed by measuring localized phosphorous high-energy metabolite concentrations by non-invasive in vivo P-31 MR spectroscopy. Regional concentrations of the P-31 containing metabolites of ATP, ADP, PCr (Adenosine triphosphate, adenosine diphosphate and phosphocreatine, respectively) are acquired along with inorganic phosphates as well as fructose and glucose phosphates, as well as fructose and glucose phosphates.

Magnetic resonance distinguishes amongst the originating metabolic compounds from the metabolic byproducts by virtue of the differing chemicals shifts between the originating and resulting species. The difference in a chemical's shifts is caused by molecular environment of the labeled nuclei, and the chemical's shifts are small in relation to the magnetic resonance field of view for either magnetic resonance imaging or magnetic resonance spectroscopy.

According to the invention, ATP and PCr levels in living and metabolically active cells are significantly different from dead or dying cells, which will contain larger than normal levels of inorganic phosphates, and will display an abnormal intracellular pH, measured as the chemical shift differences between PCr and the inorganic phosphate P 31 signals. The use of magnetic resonance to visualize differing chemicals shifts is the basis for MRI and MRS and as such is existing art, but what is unique is the use of chemical shift differences, particularly shifts attributable to specific compounds or labeled atoms, to noninvasively monitor local metabolic rates of cell viability.

In another preferred embodiment of the invention, local F-19 labeled metabolites (such as F-19 labeled glucose or precursors) are measured from local or volume RF-coils. F-19 labeled compounds are introduced which can be measured by MR F-19 imaging or spectroscopy. The signal strength is related to the presence and concentrations of the labeled compounds. Conversion of labeled compounds to metabolic products is observed by the differences in the F-19 frequencies of the converted products.

Similarly, there is extensive existing literature that describes the observation of differing chemicals shifts for both C-13 and for F-19 labeled compounds. However, the use of observing differences in chemicals shifts that occur due to cell viability and local tissue metabolism is unique.

According to one practice of the invention, F-19 labeled glucose are introduced together with the implanted cells. The utilization of the glucose will alter the concentrations of the various F-19 labeled conversion products. Turnover rates of F-19 products are measured and quantified. The relative concentrations of the initial F-19 labeled molecule and the subsequent metabolic byproducts can be determined noninvasively and rapidly from the MR imaging and MR spectroscopy experiment. Because of the varying chemical shifts, the individual signal intensity's of each and every metabolic byproduct can be immediately identified and labeled for each of the original metabolites and resulting metabolic byproducts.

In another embodiment of the present invention, cell viability is evaluated by measuring the alterations in tissue sodium measured by Na-23 MRI. According to the invention, intracellular sodium levels are distinguished from extracellular sodium levels using chemical shift differences from introduced chemical shift reagents to separate the extracellular sodium from the intracellular sodium. Only extracellular sodium is shifted by these reagents.

The presence of a paramagnetic shift reagent is used to move or alter the chemical shift of the extracellular sodium 23 nucleus. The intracellular sodium nuclei do not experience the presence of the chemical shift reagent and their chemical shift remains as before (note that the chemical shift reagents do not pass through the cell wall in viable cells and this leads to the increased sodium population with cell death.).

In another preferred embodiment of the present invention, cell viability is also evaluated by assessing the intra- and extracellular sodium based on the TI and T2 relaxation times of the two constituents. The difference in chemical shift due to the extracellular sodium from the intracellular sodium can be directly observed and related to a local concentration measured. If there is no intracellular sodium, this indicates that the cell is no longer viable and that the local environment is just extracellular sodium. In this case, the absence of an intracellular sodium signal would indicate a lack of cell viability and the presence of cell lysis.

According to the methods of the invention, these MR changes can be detected by a volume coil surrounding the tissue or by introduction of a local multi-tuned MRI RF-coil arrangement. The local MR coil can be attached to the local infusion catheter. This can also be connected to local pH meters, glucose and other meters, local gas sensors, video monitors, optical detectors, and chemical and other sensors.

Other observation techniques may also observe concentration changes aside and separate from MR. The use of local catheter containing the local MR coil could also contain other tissue sensitive sensors such as gas sensors, optical detectors, video monitors, thermal sensors, semiconductor absorption/adsorption detectors, as well as other sensors. The sensors noted may not be as sensitive to labeled compounds as MRI. Other tissue specific changes may be observed by the use of the non-MR monitors.

There are numerous types of sensors available that can be used directly to sense factors resulting from increased activity or decreased activity of implanted cells in a region of the body. Any form of sensor may be used to qualitatively and/or quantitatively determine cell survival rates by measurement of ancillary or directly measurable results (e.g., metabolic alteration) from cell survival, cell propagation, and/or cell death.

Viewed from another aspect, the invention provides methods for assessing blood flow and perfusion in the region of the cell implant, which can also be an indication of local cell viability. In one other embodiment of the invention, tissue blood flow in the region of the cell implant is assessed by measuring labeled H2O water (H217O water which shortens water proton T2 by MRI). The decay of signal is a measure of the local blood flow. The in vivo signals of the labeled water are altered with blood flow. Tissue bloodflow in the regions of the cell implant may be assessed by measuring labeled O-17 (i.e., O17 water or 17O) water. Because the presence of O17 water reduces water proton T2 relaxation times surrounding the O17 nucleus, this is directly observed in T2-weighted MR water proton images, and any change in the local water proton T2 relaxation times would be indicative of a change in the local blood flow. Thus for example, an increase in local blood flow caused by cell viability would alter the water proton T2 relaxation times in a manner different from the local blood flow provided by a nonviable cell population. A local tissue environment with no cell viability would result or would be characterized by little or no cerebral blood flow and thereby no shortening of water proton T2 relaxation times. The water proton T2 relaxation times can be directly observed as a signal intensity or as an image intensity on a T2 weighted proton magnetic resonance image. It can also be quantitated as a change in the measured water proton T2 relaxation time.

According to one aspect of the invention, the labeled water may be introduced via the infusion or implantation catheters or can be introduced at a later time. In another embodiment of the invention, local tissue blood flow is monitored by MR perfusion imaging following infusion of TI or T2* shortening agents, including especially MR visible paramagnetic, superparamagnetic, or non-magnetic contrast agent.

In another particular embodiment, local blood flow is measured in the cell implant or in the vicinity of the cell implantation by the local introduction of hyperpolarized Xenon gas (by Xe-tuned MRI RF coils). The elimination of the Xe gas and the decay of the Xe signal is a measure of the local blood flow from that region. The establishment of a viable cell colony, and the subsequent development of a capillary bed needed to supplement the cell colony results in an increase in local blood flow which can be then monitored using a variety of magnetic resonance experiments. The level of death of local cells can be assessed by the relative changes in the local blood flow to and from that region. The measurement(s) used in assessing local blood flow can be acquired together with the data and information used to assess local metabolic changes as well as with tissue relaxation time in diffusion changes in one integrated examination to provide a broad range of data to provide highly accurate measurements to determine cell survival as well as other operation environment factors.

In another embodiment of the invention, local blood flow is measured through the use of optically-active dyes and coloring agents by monitoring the concentration time changes by an optical probe. Optical imaging is shown in U.S. Pat. No. 5,919,140 (although not specific for observing survival/death of cells), and radiation observation is also known (e.g., U.S. Pat. No. 5,928,625). U.S. Pat. No. 5,871,946 shows a non-fluorescent first state when joined to said leaving group, and a leaving group is provided being in a fluorescent second state excitable at a wavelength above 450 nm when said leaving group is cleaved from said indicator group by said enzyme for a period of time sufficient for said assay reagent to be transferred into said cell and for said leaving group to be cleaved inside said cell from said indicator group by said enzyme wherein said assay compound has sufficient purity so that fluorescence of impurities in such assay compound is less than auto-fluorescence of the metabolically active whole cell;

exposing said cell to light having a wavelength above 450 nm; and

measuring fluorescence of said cell to determine the activity of the enzyme.

Local bloodflow may also be measured through the use of optically active dyes and coloring agents by monitoring the concentration time changes from an optical probe that could be incorporated together with her separate from a local MR RF coil in the catheter. By observing light at a particular wavelength, the signal intensity of the light may be altered by the transit of an optically sensitive dye or coloring agents present in the blood pool. This is not a question of fluorescence, but rather a change in signal intensity of transmitted or reflected light coming from the tissue or being altered by the passage of a optically active dye or coloring agents passing or transiting through the circulation system. This idea can be greatly expanded to include any number of optically active dyes or coloring agents as well as any number of visible or infrared wavelengths. The use of bioluminescence or fluorescence can also be used

The decay of the optical agent or optical signal as local blood flow carries the agent or compound from the cell colony.

It is one aspect of the present invention to provide an MR imaging method for the local and controlled evaluation of tissues of living cells and cell suspension as or after they are artificially grown into a functioning organ that can produce a biologically active factor without the imaging method causing trauma to the tissue or organ. This technology is also useful for evaluating the potential tissue viability of growing or grown complete and functional organs (as from cell seed growth of completely functional organs). These techniques can thereby be used as part of a non-invasive screening methodology to establish and even quantify levels of performance of tissues and organs developed in vitro in order to establish the likelihood of success and the quality of individually grown organs based upon non-invasive measurements of organ performance described herein.

A second aspect of the invention is to provide an imaging means for quantitating the number of cells implanted into a tissue in a human body.

A third aspect of the present invention is to provide an imaging means for quantitating the number of living cells implanted into a tissue in a human body.

A fourth aspect of this invention is to provide an MR imaging means for quantitating the number of cell-cell membrane contacts in a cell implant in a tissue in a human body.

A fifth aspect of the present invention is to provide an MR imaging method for quantitatively determining the apparent diffusion coefficient in a population of living cells implanted into a tissue in a human body.

A sixth aspect of this invention is to provide an MR method for quantitatively determining the pH and fluid-electrolyte parameters in a population of living cells implanted into a tissue in a human body.

A seventh aspect of the invention is to provide an MR method for quantitatively determining the phosphorus and water proton metabolites in a population of living cells implanted into a tissue in a human body.

An eighth aspect of the present invention is to provide an MR imaging means for quantitating the functional capillary density of the tissue region contiguous with the cell implant.

A ninth aspect of this invention to provide automated computer software methods for modeling and monitoring cell implant therapy, specifically for data collection related to monitoring cell viability following implants.

### BRIEF DESCRIPTION OF THE DRAWINGS

No Figures are present in this Application

### DETAILED DESCRIPTION OF THE INVENTION

As cells are introduced into a tissue for purposes of effecting therapy to a patient, it becomes critical to monitor the status of the cells and their assimilation and integration into the environment. Among the hallmarks of cell growth and proliferation that can be assessed according to the present invention (as by MRI) include:

1. The presence of metabolism and increases therein as cells replicate. To do this, we can (among other techniques) measure: local lactate levels (measured from proton spectroscopy), local glucose turnover (monitored by C-13 labeled glucose), local phosphorous high-energy metabolite concentrations (measured by P-31 MR-spectroscopy), local F-19 labeled metabolites (such as F-19 labeled glucose or precursors), alterations in tissue sodium (measured by Na-23 MRI), changes in the conversion rates of O₂ gas to H₂O water (which can be measured by 17O₂ gas conversion to H₂17O water which shortens water proton T2 by MRI).

These changes can be detected by a number of mechanisms, including but not limited to a volume coil surrounding the tissue or by introduction of a local multi tuned MRI RF coil arrangement. The local MR coil can be attached to the local infusion catheter. This can also be connected to local pH meters, glucose and other meters, local gas sensors, video monitors, optical detectors, etc.

2. The presence of blood flow or increases in blood flow as vascular supply is developed can be measured. To do this, we can measure: labeled H₂O water (H₂17O water which shortens water proton T2 by MRI), local blood volume and flow by contrast agent infusion of TI- or T2*-shortening agents, or by the local introduction of hyperpolarized Xenon gas (by Xe-tuned MRI RF coils). The use of optically-active dyes and coloring agents can also be used by monitoring the concentration-time changes by an optical probe.

3. The presence of anisotropic water diffusion as neuronal development occurs can be measured. To do this, we can measure the fractional anisotropy or any water proton diffusion indices available to show that water movement and diffusion is being altered or oriented by the growth or presence of neuronal growth, by presence or growth of myelin (or by demyelination). As tissue matures, cells orient and water is thereby oriented in its mobility. In this way, if cells form patterns or synapses, the presence of water motional anisotropy can be assessed and measured.

4. The presence of local depolarization in developing neurons and neurofibrils is measured. To do this, we can measure the local fractional anisotropy or any water proton diffusion indices available to show that cell depolarization (which is created as the electrical potential is created along a neuron or synapse) is occurring. The presence and firing of synapses will create local transient depolarization in developing nerves and neurons. The depolarization will change the local water anisotropy and thereby change the water movement and diffusion. As the neurons develop, the presence of water motional anisotropy created during the depolarization of the cell or nerve firings can be assessed and measured.

5. The evolution of certain metabolites needed for replication can be measured. These metabolites include choline, NAA, GABA, etc. Monitoring these building blocks provides assessment of cell maturation and growth. To do this, we can measure the local concentrations of choline, NAA, GABA, phosphocholine, creatine, etc. This can be done from volume MRI RF

coils surrounding the tissue or from the introduction of local MRI RF-coils.

6. Increases in local tissue density and cell populations can be measured. To do this, we can measure the water proton diffusion in the local tissue. As the cells grow and proliferate, the local density will increase, the local water content will decrease and the water diffusion coefficient measured by diffusion

weighted MRI will decrease.

7. Other characteristics or properties that can be measured include, for example, local tissue density and cell populations, local electrical activity, local oxygenated/deoxygenated hemoglobin and changes in the local T2* reflecting the alterations in tissue oxygenation, changes in the vascular reserve and response to oxygenation stresses, tissue fluorescence and bioluminescence, tissue fluorescence and bioluminescence, electrical impedance, local tissue temperature.

8. Increases in local electrical activity as could be monitored by EEG can be measured. The local electrical firings can be monitored by local EEG electrodes introduced by image guided catheters or from EEG electrodes located outside the tissue.

9. Changes in the local oxygenated/deoxygenated hemoglobin and changes in the local T2* reflecting the alterations in tissue oxygenation can be measured. It is clear that cell proliferation will increase local metabolism, creating a demand for vascular autoregulation, and delivering local arterially blood, which will in turn increase the local T2*. Thus can be measured by monitoring the local T2* changes from volume MRI RF coils outside the tissue or by local MRI RF-coils introduced by image-guided catheters.

10. Changes in the vascular reserve and response to oxygenation stresses can be measured. The development of local blood capillaries and a vascular bed will develop arterioles which are sensitive to local carbon dioxide concentrations. If the CO₂ is increased by infusion of the gas or by an oxygenation stress (such as breath holding or hypoxia or hypercapnia), then the autoregulation can be assessed by blood flow or perfusion MRI, by T2* imaging or by changes in local lactate or metabolic activities.

11. Changes in tissue fluorescence and bioluminescence can be measured. The development of cells into tissues will alter the inherent cellular luminescence, which can be monitored by a local optical probe or camera introduced by a image

guided catheter. This can also by measured by introducing an added luminescence probe, such as GFP (green fluorosecent pigment)), or any optical-active agent, such as food dyes or color agents.

12. Changes in electrical impedance can be measured. The development and growth of cells will change (increase) the local electrical impedance of the tissue. Since the impedance of necrosis cells or tissue falls, the increase in impedance indicates increases in the number of intact (and available) cells.

13. Increases in local tissue temperature can be measured. To do this, we can measure the local temperature using probes introduced by image-guided catheters. The local temperature will increase with increased metabolism. The establishment of blood flow will regulate the temperature to stresses. The blood flow can be indirectly assessed by local heating or cooling of a tissue by introduction of an image-guided catheter laser or cryoprobe.

H₂17O Enhanced MRI of Blood Flow Using 17O Decoupling Introduction

The use of oxygen-17 gas and labeled water have been studied for some time as a means of measuring perfusion and oxygen metabolism in vivo. H₂17O in particular is a freely diffusable tracer attractive for in vivo blood flow measurements. Disadvantages include: low sensitivity of 17O NMR, low natural abundance of H₂17O (0.035%), high cost of 17O enriched compounds. Recently, improved sensitivity has been reported in imaging applications using the T2-relaxation effect of H₂17O on protons at steady state concentration (A. Hopkins, E. Haacke. et al. Magn. Reson. Med. 7:222-229 (1988).) and as a bolus injection for CBF measurement (K. Kwong, A. L. et al. Magn. Reson. Med. 22:154-158 (1991).). 17O shortens proton T2 via an exchange modulated scalar coupling through the hydrogen bond; this interaction can be suppressed by irradiating at the 17O frequency (i.e. decoupling) while protons are undergoing T2 relaxation. This effect has been exploited to measure 17O concentration by comparing the 1H signal in a spin echo sequence with or without 17O decoupling (I. Ronen, G. Navon, Mag. Reson. Med. 32:789-793 (1994)) and also been demonstrated for MR imaging using a double tuned coil (R. Reddy, A. Stolpem, et al. Mag. Res. Img. 14:1073-1078 (1996).).

In another application of the technology within the scope of the generic disclosure herein, the introduction of 17O₂ gas to a tissue can be used to probe the ongoing metabolic state by monitoring the conversion of the gas to H217O water, which then shortens the water proton T2 values. In this way, the gas can reveal the presence of metabolism in a tissue. While this approach has been patented earlier (Arai, et al.), the means of introducing the gas and the method of use for cell viability monitoring has not been explored adequately.

The technology will be then to introduce either the 17O₂ gas to a tissue by means of a proprietary cranial insertion device (the “Navigus™ of ImageGuided Neurolgics, Inc., or derivations thereof), or by introducing the H217O water for monitoring cell viability by measuring blood flow. The infusion of the gas or the water by means of a interstitial catheter has many uses:

1. The gas can be used to assess ongoing metabolism, since the conversion to water is necessary to observe a decrease in proton T2. Viable cells will possess metabolism.

2. The conversion of the gas to water can be used to measure the local blood flow by monitoring the dissipation of the proton T2 shortening effect as the water is removed from the tissue by the local blood flow.

3. The local blood flow can also be measured by infusing H₂ 17O water directly. We can assume that cells are becoming viable if the local blood flows increase as local metabolism increases.

As noted above, recent technological developments have been announced in tissue and organ engineering, wherein new cells have been combined with an organ support structures (that is, underlying physical structure of organs without functioning organ tissue, such as the skeletal support for a heart without muscles, without living functionality (e.g., a heart beat, hormone producing functionality, oxygen exchange functionality, nutrient absorption and utilization functionality, and the like), such that the organ support structures oe cytoarchitecture of tissues provide an engineering scaffold for the new cells. It is a likely scenario in the production and growth of independent and complete organs by this technology that defects will occur in the growth of individual organs, and that these defects might appear as localized defects wherein certain tissue components of the developing organ will manifest abnormal or suboptimal characteristics, including, for example, abnormal metabolic or functional activity. It is also likely, as with certain in vitro fertility treatments, that to assure or increase the potential for eventual transplant success, multiple organs will be grown at the same time and only organs with demonstrable metabolic and functional viability will be selected for implantation. A need clearly exists for useful, non-invasive screening methods for identifying tissues and organs with demonstrably viable metabolic and functional viability according to measurable parameters. In particular, scientists have worked for years for ways to grow body parts, including specialized tissue and organ components. For example, considerable effort has been focused on developing heart valves as an alternative to the plastic or animal valves that wear out after being implanted in humans.

An estimated 5 million people live with heart failure and about 550,000 new cases are diagnosed each year in the United States. Approximately 50,000 die annually waiting for a heart donor. Other organs, including liver, pancreas, adrenals, are also in short supply for transplants. These newer cell seeded organ growth technology can enable medical practitioners to grow complete organs from cells derived from patients targeted to receive the organs, which would offer the potential for nearly completely eliminating organ rejection issues from the transplant and provide a specific time window for availability of organs.

In an already proven example of seeded organ growth, the University of Minnesota took the hearts from eight newborn rats and removed all the cells. Left behind was a gelatin-like matrix (a non-functional support structure) shaped like a heart and containing conduits where the blood vessels had been. Scientists then injected cells back into this scaffold—muscle cells and endothelial cells, which line blood vessels.

The muscle cells covered the matrix walls and lined up together, while the endothelial cells found their way inside to coat the blood vessels. Then the hearts were stimulated electrically.

Within two days, microscopic contractions were observed, and by seven to eight days there were contractions large enough to see with the naked eye. The tiny hearts could pump liquid at about one-fourth the rate of a normal fetal rat's heart. It has been suggested that a similar process could work with either human hearts from cadavers or pig hearts, with their cells stripped off and replaced by cells from the person needing a heart transplant to avoid rejection.

The next step to be taken by the University of Minnesota is to take a pig heart, strip away the cells and repopulate it with cells from a pig to see if it will work in the larger heart. The University has already reported that the researchers are working on reseeding cells into other organs, including lungs, liver and kidneys. U of M researchers say they have succeeded where others failed by using the framework of a dead heart. The process is much easier than creating a heart de novo.

The team created its new heart by flushing the dead organ to remove every existing cell. What remained was a snow white shell that is essentially the same material as the gristle on a steak. This created a cytoarchitectural template or scaffold that looks like a heart and that scaffold can then be used start to build a new organ. In research circles this work is called tissue engineering.

The team then collected new living heart cells from a donor animal and re-seeded the empty organ scaffolding. The seeded heart cells in there began to know how to line up, the begin to know how to go to the blood vessels and form new blood vessels and line the blood vessels again. The team hooked a pacemaker up to this and the heart cells started to beat and contract and over time it matured.

As the organ matured, it grew layers of muscle in the heart walls. It also appeared to get stronger when researchers increase the volume of blood flowing through the organ. And the heart continued to beat even after the pacemaker is turned off.

One challenge in the development of the technology to an applicable state is making sure the right cells grow in the right places in the heart. An issue is how to get those cells to home in where you want them to home in on proper location and function. As the different cells produce different by-products and chemically alter their environment in different ways, the technology used herein can be used to assure or at least provide repeatable evidence that appropriate cells are in appropriate locations and performing at appropriate levels.

The technology may be alternatively described as a method for indicating viability of transplanted progenitor or stem cells in an organ engineering environment, the method being performed with a medical device that supports at least one sensing function. The method may comprise:

non-destructively observing a region of the growing tissue or organ where cells have been placed;

sensing a property within said region of the organ that is indicative of cell viability or nonviability of the cells; and

using data from sensing said property within said region to indicate cell viability from the cells on the scaffolding or which tissue cytoarchitecture constitutes the scaffolding within the organ wherein said cell viability is indicated by a property in cell chemistry resulting from an event selected from the group consisting of cell activity, cell inactivity, cell growth, cell death, specific cell function, specific cell dysfunction, volumetric expansion of cell population, and volumetric decrease of cell population.

Another description of technology described herein is as a method for indicating viability of transplanted progenitor or stem cells in an organ engineering environment, the method being performed with a medical device that supports at least one sensing function. The method comprises:

non-destructively observing a region of the organ where cells have been positioned;

sensing a property within said region of the organ that is indicative of cell viability or nonviability of provided cells grown on the scaffolding; and

using data from sensing said property within said region to indicate cell viability from the provided cells grown on the scaffolding within the region, wherein said non-destructively observing comprises magnetic resonance imaging and wherein said cell viability is indicated by a property in cell chemistry resulting from an event selected from the group consisting of cell activity, cell inactivity, cell growth, cell death, specific cell function, specific cell dysfunction, volumetric expansion of cell population, and volumetric decrease of cell population.

The sensing of a property within said region of a patient that is indicative of cell viability or nonviability of transplanted progenitor or stem cells grown in a culture may use data from sensing said property within said region to indicate cell viability from a transplant of progenitor or stem cells grown in a culture within the region wherein said property is monitored by observation of at least one parameter selected from the group consisting of local lactate levels, local glucose turnover, local phosphorous high-energy metabolite concentrations, local F-19 labeled metabolites, alterations in tissue sodium, and changes in the conversion rates of O₂ gas to H₂O water, or use data wherein the property is monitored by observation of at least one parameter selected from the group consisting of local lactate levels, local glucose turnover, local phosphorous high-energy metabolite concentrations, local F-19 labeled metabolites, alterations in tissue sodium, changes in the conversion rates of O₂ gas to H₂O water, proton spectroscopy, monitoring of C-13 labeled glucose, monitoring by P-31 MR spectroscopy, monitoring of local F-19 labeled metabolites, monitoring of Na-23 levels, and monitoring of 17O₂ gas conversion to H₂17O water. The medical device may include at least one element selected from the group consisting of a volume coil surrounding the tissue and a local multi-tuned MRI RF coil.

The method for indicating viability of cells grown as complete transplantable organ during growth of the organ or after growth of the complete organ by cell growth over a cell rejection inert scaffolding, the method being performed with a medical device that supports at least one sensing function may also be described as comprising:

non-destructively observing a region of the organ where cells have been provided on the scaffolding and cells are growing, e.g., on the tissue cytoarchitecture that may form the scaffolding;

sensing a property within said region of the organ that is indicative of cell viability or inviability of provided cells; and

using data from sensing said property within said region to indicate cell viability within the organ wherein said property comprises blood flow or changes in blood flow as vascular supply is developed.

One of the significant problems with delivering cells directly into the brain is to assure that the cells are accurately delivered to a target location and thereafter remain viable. In the method of the present invention, MRI navigation procedures are used to guide a MR compatible access device and a catheter containing the cell implant to a target location in the brain or other tissue. Following MRI-guided positioning of the catheter tip at the target location, delivery of the cell implant is monitored using an imaging system particularly high-resolution imaging systems, and most particularly high-resolution MR imaging, alone or in combination with optical or other imaging methods Further aspects in the method of the invention include the use of high-resolution MRI methods to non invasively evaluate the viability of implanted cells.

MR need not be used in combination with other imaging technologies for this imaging method. Non-imaging detection technologies may also be used optionally with the MR imaging. For example, MR may be used alone or in combination with an optical or pH or gas detection/observation methodologies, or each individual methods may be used alone. The idea of an MR guided catheter to establish or plant a colony of implanted cells can be expanded logically to include any number of sensors introduced through the MR guided catheter. The catheter may include a local MR RF coil, a local gas sensor, the local pH meter or any local factor-sensitive or tissue measuring device. Separate from the above, one may also monitor the magnetic resonance imaging changes or the magnetic resonance spectroscopy changes through the use of a larger external coil separate from any local catheter are coil. The disadvantage in the larger external coils is that of signal-to-noise and sensitivity. The local radio frequency coil for MR imaging our spectroscopy increases the resolution and sensitivity of the non-invasive assessment of the viability of implanted cells.

According to the present invention, MRI navigation procedures are used to guide an MR-compatible intracranial access device, such as described in U.S. patent application Ser. Nos. 08/919,649 and 08/856,664, which is used in combination with a specialized catheter, such as disclosed in U.S. patent application Ser. No. 08/856,664, containing the cell implant to reach a target location in the tissue. In one preferred embodiment of the invention, if the catheter trajectory requires a curved path, a combination of magnetic stereotaxis (MSS) and MRI navigation methods are used, as previously disclosed in U.S. patent application Ser. Nos. 09/131,031 and 09/174,189. Following MRI-guided positioning of the catheter tip at the target location, delivery of the cell implant is monitored using high-resolution MRI as disclosed in U.S. patent application Ser. No. 08/857,043. According to the present invention, drug delivery may also be performed by infusion via convection efflux from a single peripheral lumen, such as disclosed in U.S. patent application Ser. No. 08/856,664, or via a multi-port and/or multi-lumen configuration in order to facilitate broad spatial distribution of the drug within the region of the cell implant.

A further feature of the present invention is that cell delivery is achieved through a central barrel of the catheter, whereas the surrounding peripheral ports of the catheter house any one of a combination of physiologic probes. As disclosed in U.S. patent application Ser. Nos. 08/856,894 and 09/131,031, the probes could include intracranial pressure probes, optical fibers and/or optical fiber bundles configured for conveying illumination and/or optical signals to and from the target tissues, iontophoresis probes, thermometry probes, blood-sensing probes, chemical probes, vacuum lines, fluid delivery tubes and conduits, guidewires, fixed and adjustable-tipped steering probes and wires, electric and magnetic field-sensing probes, electrodes and applicators, gene analysis chips and “lab-on-chip” structures, biopsy devices, tissue and coil implantation devices, cryogenic probes, cauterizing probes and tips, stents, coils, angioplasty balloons and devices, radioactive sources, magnetic and electric field sources, integrated circuits and other electronic devices.

In a method of the invention, the cells may include secretory cells which have been isolated from natural sources, or have been genetically engineered to produce neuroactive factors, growth factors, cytokines, antibodies, extracellular matrix components or neurohormonal agonists (peptides or bioactive amines), precursors, active analogs, or active fragments. In a preferred aspect of the invention, the cell is an allograft or a xenograft. Preferred cell types for specific therapies may include those dopamine secreting cells from the embryonic ventral mesencephalic brain, from neuroblastoid cell lines, or from the adrenal medulla. Any cells that have been genetically engineered to express a neurotransmitter or its agonist, precursor, derivative, analog, or fragment which has similar neurotransmitter activity, or bioactive macromolecular factors (see above), can also be used to practice this invention. For example, genetically engineered fibroblasts or other cell types may be used.

As used herein, the term “biologically active factors” has the same common meaning as described in U.S. Pat. No. 5,487,739, namely, neurotransmitters, neuroactive analgesic factors, as well as precursors, agonists, active analogs, and active fragments. Also included are proteins, nucleic acids and other macromolecules having biologic activity, as well as agents, which might be infused for their physical or chemical properties. Examples of biologically active macromolecules could include growth factors, cytokines, antibodies, hormones, oligonucleotides, modified long DNA constructs (synthetic vectors), glycoproteins and glycolipids. Examples of agents which might infused for their physical properties could include radiographic contrast agents or reagents to enhance the in vivo detection of implanted cells or the products they have been engineered to produce. Also expressly included are cells that secrete neuroactive factors and hormones, including “growth factors” such as described in U.S. Pat. No. 5,487,739 to Aebischer et al.

Viewed from one aspect, the present invention provides MR imaging methods for evaluating the viability of a cell implant over days, weeks, months or years. In the method of the invention, high-resolution MRI methods, most preferably diffusion tensor imaging, as disclosed in U.S. Pat. Nos. 6,026,316 and 6,061,587 co-authored by the present inventors, are used to evaluate the viability of the cell implant following the delivery of the cell implant into the target location in the brain. MR diffusion coefficient imaging, as disclosed in those U.S. patents, is used concurrently to evaluate fluid electrolyte homeostasis in the extra-cellular and intra-cellular fluids contiguous with the cell implant.

According to some aspects of the methods of the generic invention disclosed herein, cell viability may be assessed by monitoring the presence of anisotropic water diffusion. As the implanted cells organize into coherent patterns or populations, orientational influences are imposed on the mobility of adjacent water protons giving rise to fractional anisotropy changes that can be measured with MR. Furthermore, in a method of the invention, cell viability may also be assessed by monitoring the increases in local tissue density by measuring the water proton diffusion in the local tissue. As the cells grow and proliferate, the local density will increase, the local water content will decrease, and the water diffusion coefficient measured by diffusion-weighted MRI will decrease. Thus, according to the invention, cell viability can be assessed by measuring the apparent diffusion coefficient of the ADC (Apparent Diffusion Coefficient) by non-invasive proton diffusion MRI.

The apparent diffusion coefficient of the water proton or the ADC can be accurately measured by non-invasive proton diffusion MRI. This can be quantitated and compared with previous or subsequent separate assessments. Cell viability can be assessed from the quantitative ADC. In fact, cell proliferation leads to decreases in the water proton ADC by virtue of the presence of increased barriers to water mobility, measurable as decreases in the ADC. In this is not a measure of water content but rather a measure of the mobility of the water proton to its microenvironment. As such the ADC may be accurately quantitated and compared over time. The increases in local tissue density obtained by measuring the water proton ADC in the local tissue is a method of assessing the number of viable cells within the voxel by noting and quantitating the ADC, which is sensitive to and his altered by the presence of increased cellular barriers.

In another embodiment of the invention, the viability and functional assimilation of the implanted cells may be assessed by monitoring changes in the resting membrane potential of cells in the cell implant. Electrical potentials, most particularly depolarization or hyperpolarization, arising in or contiguous with the cell implant will give rise to local fractional anisotropy or other water proton diffusion indices which, according to the methods of the invention, can be detected by MR. Changes in membrane potential will change the local water anisotropy and thereby change the water movement and diffusion. In another particular embodiment of the invention, cell implant viability is assessed by measuring the increases in local electrical activity by local EEG electrodes introduced by image-guided catheters or from EEG electrodes located outside the tissue.

In a further embodiment of the invention, cell viability may also be assessed by measuring changes in electrical impedance in the region of the cell implant. The development and growth of cells will increase the local electrical impedance of the tissue. The increase in impedance provides a measure of the increased number of intact functional cells in the implant.

The use of electrical impedance within the region of the cell implant may well be variable by placement or with changes in the placement of the impedance device. Practically, a general or averaged measurement could be derived by altering the placement of the catheter in numerous or repeated experiments or procedures. According to certain specific practices within the broad scope of the invention, the viability of implanted cells may also be assessed by measuring the evolution of certain MR-visible metabolites needed for cell replication. These metabolites include choline, N acetylaspartate (NAA) and gaba-aminobutyric acid (GABA). In one embodiment of the invention, the local concentrations of choline, NAA, GABA, phosphocholine, creatine are measured from volume MRI RF-coils surrounding the tissue or from the introduction of local MRI RF-coils. Metabolite signals are observed from non-invasive proton spectroscopy or from localized proton spectroscopic imaging.

Proton MR spectroscopy is highly sensitive to a number of MR visible metabolites used or needed for cell viability and replication. These metabolites may be measured and their relative concentrations quantitated from either external volume MRI RF coil surrounding the tissue or from measurements taken from the local RF coils. These metabolites are easily distinguished from one another by virtue of their differing chemical shifts. Thus any increase in the lactate signal, measured at the proton lactate chemical shift, would indicate and increase in cellular metabolism. If the lactate signal intensity builds overtime, this indicates cell death. Because the individual metabolites had differing chemical structures and differing chemical reactivities, one can design a local semiconductor sensor array, introduced by the catheter, which would be reacted to the individual metabolites and thereby serving as a semiconductor sensor for concentrations of one or more components or products. Within the semiconductor sensor array, compounds within the array would react with or adsorb/absorb tissue metabolites and could then be used to alter light emission, bioluminescence, or fluorescence. The increase in local tissue lactate would increase the presence or decrease the presence of a reactive/absorptive compound within the semiconductor sensor array and thus alter the light emission or reflectance or transmission from the array. One could greatly expand this alternative, exploring the use of local semiconductor sensor arrays with tissue metabolite specific compounds within the array that could react with local tissue metabolites and cause some measurable change in the semiconductor array that could be detected through the use of MR, optical, gas, pH, temperature and other sensing systems. Given the sensitivity and the convenience of MR spectroscopy, increases in tissue lactate and/or decreases in any of the other metabolites or metabolic byproducts would lead to a similar assessment.

In another embodiment of the invention, cell viability is assessed by measuring changes in the local hemoglobin oxygenation state together with changes in the local T2* reflecting the alterations in tissue oxygenation. Cell proliferation will increase local metabolism, thereby increasing local vascular autoregulation, and preferentially delivering fully oxygenated arterial blood, which will in turn increase the local T2*. In the method of the invention, the increased delivery of oxygenated blood can be monitored by monitoring the local T2* changes from volume MRI RF-coils outside the tissue or by local MRI RF-coils introduced by image-guided catheters.

The process of cell growth and for cell death will alter many if not all of the MR observable processes mentioned above. By measuring and quantitating tissue relaxation times diffusion times relative for absolute cell metabolite concentrations, one may compare either longitudinal change in a relative signal intensity or a longitudinal alteration in a quantifiable measurable quantitative assessment of the rate of cell growth and or death. By quantitating or measuring a highly specific tissue metabolite or characteristic, this series of images acquired over long duration longitudinally need not be highly comparable. One can for example immediately compare a bulk measurable quantity or performed voxel by voxel comparison or any sort of statistical comparison of measurable values over time, either visually or by computational methods (as described above).

In the method of the invention, cell viability may also be assessed by measuring changes in the vascular reserve and response to oxygenation stresses. As the population of cells in the cell implant grows over time, the local microvasculature will develop increasing numbers of arterioles which are sensitive to local carbon dioxide concentrations. According to the invention, if the CO₂ is increased by infusion of an exogenous gas or by an oxygenation stress (such as breath-holding or hypoxia or hypercapnia), local vascular autoregulation can be assessed by blood flow or perfusion MRI, by T2* imaging or by changes in local lactate or metabolic activities.

Many of the measurable materials or conditions, such as lactate concentrations, will need to be comparable over time. The use of the MR guided catheter is a convenient but not critical parameter. Note that highly specific added anatomically accurate MR morphological images may be acquired together with proton diffusion relaxation times or metabolite measurables. Thus quantitative measurements may also be roadmaps to local tissue morphology, thus assuring consistent site comparisons. In other words, the lactate concentrations or the proton diffusion coefficient can be related to local tissue morphologies. In this way cell viability of a colony placed within the hippocampus for example can be related to or overlaid upon the anatomic MR image.

Further in the method of the invention, cell viability may be assessed by measuring changes in tissue fluorescence and bioluminescence. In a preferred embodiment of the invention, the development of cells into tissues will alter the inherent cellular luminescence, which is monitored by a local optical probe or camera introduced by a image-guided catheter. In a particularly preferred embodiment, tissue luminescence can also by measured by introducing an added luminescence probe, such as probe which is optically sensitive to Green Fluorescent protein (GFP), or any optically active agent, such as food dyes or color agents. U.S. Pat. No. 5,928,625 uses green fluorescent dyes to observe cells, but without any mechanism for qualitatively or quantitatively assessing cell survival.

The use of a local fluorescence probe sensitive to GFP or green fluorescent protein would be a useful but not critical addition to the practice of this technology. While the MR guidance is clearly a preferred integral aspect to the scope of the invention, it is the MR measurables that provide non-invasive and quantifiable measures of cell viability that is a major benefit of this invention.

In a further embodiment of the method of the invention, cell viability may be assessed by measuring changes in local tissue temperature using, as one example, probes introduced by image-guided catheters, or from temperature-sensitive phase changes in the proton MRI signal. The local temperature will increase with increased metabolism reflecting the underlying viability of the implanted cells.

Viewed from another perspective, the invention provides a method to evaluate the functional capillary density of the cell implant longitudinally over days, weeks, and months to confirm that the cell implant has adequate perfusion to sustain its viability and growth. In the method of the invention, MR first-pass perfusion imaging, such as disclosed by U.S. Pat. Nos. 5,190,744, 5,494,655, and 5,833,947 to Rocklage et al. are used to measure perfusion in the volume of brain tissue occupied by the cell implant in ml/gram tissue/minute. The average distance of any cell in the implant to a functional perfusing capillary can be estimated by knowing the number of cells implanted and the volume of the cell implant. The functional capillary density can then be expressed as the number of perfusing capillaries per 1000 cells/per minute. Deficiencies in perfusion can be established by measuring a low functional capillary density, whereupon the implant can be treated by the local intracranial administration of angiogenic or other neurotrophic drug agents to improve perfusion and long-term cell viability.

Viewed from another perspective, the present invention also provides a method to measure perfusion and oxygen metabolism of the cell implant in vivo by using oxygen-17 gas and labeled water. H₂17O is a freely diffusible tracer which has been used for in vivo blood flow measurements. While H₂17O has several disadvantages, including low-sensitivity of 17O NMR, low natural abundance of H217O (0.035%), and high cost of 17O enriched compounds, recent publications in the medical literature (e.g. Kwong, et al., Magn. Reson. Med. 22:154-158, 1991; Hopkins et al., Magn. Reson. Med. 7:222-229, 1988) have reported improved sensitivity of H₂17O in imaging applications using the T2-relaxation effect of H₂17O on protons at steady state concentration and as a bolus injection for CBF measurement. O17 shortens proton T2 via an exchange modulated scalar coupling through the hydrogen bond; this interaction can be suppressed by irradiating at the O17 frequency (i.e. decoupling) while protons are undergoing T2 relaxation. This effect has previously been exploited to measure 17O concentration by comparing the 1H signal in a spin-echo sequence with or without 17O decoupling (e.g. Ronen et al., Mag. Reson. Med. 32:789-793, 1994) and for MR imaging using a double tuned coil (e.g., Reddy et al., Mag. Res. Img. 14:1073-1078, 1996).

In one embodiment of a method of the invention, 17O₂ gas is introduced into tissues of the brain contiguous to the cell implant in order to evaluate the metabolic state by monitoring the conversion of the gas to H₂17O water, which then shortens the water proton T2 values. The extent of T2 shortening reflects the metabolic state of the tissue, and can be measured by MR, according to the methods of the invention. While this approach has been disclosed in U.S. Pat. No. 5,479,924 to Novan et al. A method of measuring the 17O isotope content and distribution in a body by magnetic resonance imaging (MRI) by collecting first and second nuclear magnetic resonance (NMR) signals of the body under the same conditions of examination except that before one examination, the body is first irradiated with radio waves at the 17O resonance frequency, whereas before the other examination the body is not so irradiated. The difference in the results of the two examinations is measured to provide an NMR image of the 17O isotope distribution in the body.

This aspect of the present invention is distinguishable at least because it discloses an entirely different means of introducing the gas (intraparenchymal versus intravenous in the Novan patent) and utilizes a different method for assessing cell viability monitoring. The work disclosed by Novan covers several uses of 17O changes to water proton T2 relaxation times.

This embodiment of the method of the invention will now be described further by way of example with particular reference to certain nonlimiting embodiments.

### EXAMPLES

Focal cerebral ischemia was induced in young male rats by endovascular insertion of suture material into one middle cerebral artery. A femoral vein catheter was inserted for contrast agent injection. MR imaging was carried out using a 2T GE Omega system. A 25 mm 1H surface coil was placed on the rat's head, with an orthogonal 17O Helmholz coil around it. Coil isolation was improved with proton bandpass/reject filters in the 1H/17O coil cables. Multislice T2-weighted EP1 (FOV 40 mm, 64×64 1.5 mm slice, TE 90 ms) was used. Three axial slices covered the rat brain, while one slice covered tubes of 0.4% atm. and 0.9% atm. H217O used as standards. Dynamic T2-wt. EPI (TR 2s) was performed during a bolus injection of 1.0 ml of 10% atm. enriched H217O into the femoral vein. Five minutes later, a series of 128 T2-wt. images was acquired (TR 4s) with the decoupler alternately switched on and off every 16 images.

Decoupler power was applied either side of the 180° pulse. For each pixel, the cross correlation coefficient was calculated between the signal and the “on-off-on” decoupler timecourse and correlation maps generated. Pixels with high enough H₂17O concentration will yield a significant correlation. Diffusion EPI and perfusion imaging of a GdDTPA bolus was also carried out.

The results demonstrate that the use of alternating 17O decoupling can detect regional variations in H₂17O content due to cerebral pathology in vivo, even at very low concentrations. The results indicate that the method of the invention may be usefully applied to study cerebral perfusion and metabolism by monitoring uptake of 17O₂ gas or 17O labeled water.

In the treatment of Parkinson's disease, it is possible to surgically remove neural progenitor or stem cells from a patient, grow the cell in culture, insert therapeutic genes, and then replace the transfected cells back into the patient's brain. However, the ability to monitor correct cell placement non-invasively with MR imaging is not currently available. In one particularly preferred embodiment of the present invention, MR imaging is used to deliver cells which are stably transfected with a detectable marker. In a preferred embodiment, a pIRES/EGFP DNA vector, as disclosed in U.S. Pat. Ser. No. 60/082,941, carrying the Green Flourescent Protein (GFP) marker is used to establish stably transfected cell lines, and for monitoring cell delivery under MR imaging.

In another particularly preferred embodiment of the present invention, optical imaging is used in combination with MRI for cell implant localization and monitoring. Optical fibers and/or optical fiber bundles configured for conveying illumination at the appropriate wavelength for the cells transfected with GFP are used to monitor cell delivery from the catheter into the target brain tissue region. According to a study published by Phillips in Current Opinion in Structural Biology 1997, Vol. 7, pps. 821-827, “the regularity of the B-can of GFP is quite remarkable. The eleven strands of the sheet form an almost seamless symmetrical structure . . . . In fact the structure is so regular that water molecules on the surface of the can also form stripes around the surface of the cylinder . . . .” Thus, according to the present invention, cells transfected with GFP are imaged with optical methods, as well as with high-resolution MR methods using high-field (1.5 Tesla or greater) magnets and high gradient values (b>1000) applied in three orthogonal directions. The combination of optical imaging and MR imaging is used to differentiate GFP transfected cells from other cells in the tissue volume adjacent to the cell implant based on their apparent diffusion coefficient differences.

Increases in ICP (Intracranial Pressure) induced by intraparenchymal injections of liquid drug agents or following cell implants can injure tissues directly (by pressure induced cell membrane perturbations), or indirectly (by inhibiting the efficacious distribution of the drug due to tissue swelling and retarded interstitial solute transport). Thus, it is advantageous and potentially important to monitor any local and regional increases in ICP resulting from injections of liquid drug agents directly into the brain parenchyma. U.S. Pat. No. 6,537,232, co-authored by Dr. John Kucharczyk, one of the present inventors, discloses an MR-visible drug delivery device which incorporates a method and means for monitoring ICP, and to obtain near real-time information on tissue pressure changes during interventional procedures in an intra-operative MR system.

In one method of the present invention, a feedback mechanism is used to automate and optimize the monitoring of cell viability, wherein a number of physiological variables can be taken into account by the algorithm that governs the therapeutic response of the catheter system. In a preferred embodiment, physiological and metabolic data on the status of the patient (derived form other sensors on/in the body, such as, for example, probes or apparatuses which monitor tissue oxygen levels, blood flow, and other physiologic parameters) can be incorporated into the algorithm's treatment optimization process.

In another preferred embodiment of the method of the invention, the algorithm governing the patient's therapy preferably utilizes proportional-integral

derivative (PID) control functions, adaptive control functions, nonlinear control functions, multi-variable/state-space control functions, stochastic control functions and/or any other functional approach deemed appropriate for the implementation of the therapy. In all such cases, the controller could be designed to respond to changes in the patient's condition using artificial intelligence or other cybernetic techniques that would let the feedback mechanism “learn” the best way to respond to changes in the patient's physiological or anatomical status. Such techniques might employ, among other techniques, “fuzzy logic” algorithms that can function in the presence of incomplete or indeterminate data.

In the preceding detailed description of the preferred embodiments, references made to the accompanying drawings, which form a part hereof, and in which is shown by way of illustration specific preferred embodiments in which the invention may be practiced. These embodiments are described in sufficient detail to enable those skilled in the art to practice the invention, and it is to be understood that other embodiments may be utilized and that structural, logical, physical, computational, medical, architectural, and electrical changes may be made without departing from the spirit and scope of the present invention. The preceding detailed description is, therefore, not to be taken in a limiting sense, and the scope of the present invention is defined only by the appended claims and their equivalents.

What is claimed:

1. A method for indicating viability of transplanted progenitor or stem cells in an organ engineering environment, the method being performed with a medical device that supports at least one sensing function, the method comprising:
non-destructively observing a region of the growing tissue or organ where cells have been placed;
sensing a property within said region of the organ that is indicative of cell viability or nonviability of the cells; and
using data from sensing said property within said region to indicate cell viability from the cells on tissue cytoarchitecture within the organ wherein said cell viability is indicated by a property in cell chemistry resulting from an event selected from the group consisting of cell activity, cell inactivity, cell growth, cell death, specific cell function, specific cell dysfunction, volumetric expansion of cell population, and volumetric decrease of cell population.

2. A method for indicating viability of transplanted progenitor or stem cells in an organ engineering environment, the method being performed with a medical device that supports at least one sensing function, the method comprising:
non-destructively observing a region of the organ where cells have been positioned;
sensing a property within said region of the organ that is indicative of cell viability or nonviability of provided cells grown on tissue cytoarchitecture; and using data from sensing said property within said region to indicate cell viability from the provided cells grown on the tissue cytoarchitecture within the region, wherein said non-destructively observing comprises magnetic resonance imaging and wherein said cell viability is indicated by a property in cell chemistry resulting from an event selected from the group consisting of cell activity, cell inactivity, cell growth, cell death, specific cell function, specific cell dysfunction, volumetric expansion of cell population, and volumetric decrease of cell population.

3. The method of claim 2 wherein sensing a property within said region of a patient that is indicative of cell viability or nonviability of transplanted progenitor or stem cells grown in a culture; and
using data from sensing said property within said region to indicate cell viability from a transplant of progenitor or stem cells grown in a culture within the region wherein said property is monitored by observation of at least one parameter selected from the group consisting of local lactate levels, local glucose turnover, local phosphorous high-energy metabolite concentrations, local F-19 labeled metabolites, alterations in tissue sodium, and changes in the conversion rates of O₂ gas to H₂O water.

4. The method of claim 2 wherein said property is monitored by observation of at least one parameter selected from the group consisting of local lactate levels, local glucose turnover, local phosphorous high-energy metabolite concentrations, local F-19 labeled metabolites, alterations in tissue sodium, and changes in the conversion rates of O₂ gas to H₂O water.

5. The method of claim 2 wherein said property is monitored by at least one technique selected from the group consisting of proton spectroscopy, monitoring of C-13 labeled glucose, monitoring by P-31 MR spectroscopy, monitoring of local F-19 labeled metabolites, monitoring of Na-23 levels, and monitoring of 17O₂ gas conversion to H₂17O water.

6. The method of claim 2 wherein said property is monitored by at least one technique selected from the group consisting of proton spectroscopy, monitoring of C-13 labeled glucose, monitoring by P-31 MR spectroscopy, monitoring of local F-19 labeled metabolites, monitoring of Na-23 levels, and monitoring of 17O₂ gas conversion to H₂17O water.

7. The method of claim 2 wherein said medical device includes at least one element selected from the group consisting of a volume coil surrounding the tissue and a local multi-tuned MRI RF coil.

8. The method of claim 2 wherein said medical device includes at least one element selected from the group consisting of a volume coil surrounding the tissue and a local multi-tuned MRI RF coil.

9. The method of claim 12 wherein said medical device includes at least one element selected from the group consisting of a volume coil surrounding the tissue and a local multi-tuned MRI RF coil.

10. A method for indicating viability of cells grown as complete transplantable organ during growth of the organ or after growth of the complete organ by cell growth over a cell rejection inert scaffolding, the method being performed with a medical device that supports at least one sensing function, the method comprising:
non-destructively observing a region of the organ where cells have been provided on the scaffolding and cells are growing;
sensing a property within said region of the organ that is indicative of cell viability or nonviability of provided cells; and
using data from sensing said property within said region to indicate cell viability within the organ wherein said property comprises blood flow or changes in blood flow as vascular supply is developed.

11. The method of claim 10 wherein said non-destructively observing comprises magnetic resonance imaging and said property comprises blood flow or changes in blood flow as vascular supply is developed.

12. The method of claim 10 wherein said property is monitored by observation of at least one parameter selected from the group consisting of local lactate levels, local glucose turnover, local phosphorous high-energy metabolite concentrations, local F-19 labeled metabolites, alterations in tissue sodium, and changes in the conversion rates of O₂ gas to H₂O water said property comprises blood flow or changes in blood flow as vascular supply is developed.

13. The method of claim 10 wherein blood flow or changes in blood flow are measured by observation of at least one material selected from the group consisting of labeled H₂O water, contrast-agent infusion of TI-shortening agents or T2*-shortening agents, local introduction of hyperpolarized Xenon gas, or optically-active coloring agents.

14. The method of claim 11 wherein blood flow or changes in blood flow are measured by observation of at least one material selected from the group consisting of labeled H₂O water, contrast-agent infusion of TI-shortening agents or T2*-shortening agents, local introduction of hyperpolarized Xenon gas, or optically-active coloring agents.

15. The method of claim 1 wherein multiple tissues or organs from a single donor are simultaneously grown, cell viability or cell nonviability of the multiple tissues or organs is compared among each other, and a best organ is selected from among the multiple organs for implantation.

16. The method of claim 2 wherein multiple tissues or organs from a single donor are simultaneously grown, cell viability or cell nonviability of the multiple tissues or organs is compared among each other, and a best organ is selected from among the multiple organs for implantation.

17. The method of claim 2 wherein there is sensing a property within said region of a patient that is indicative of cell metabolism;
then repeating or continuing said sensing of a property over a period of time in which said property changes; and
using data from sensing changes in said property within said region to indicate cell viability in the organ cells,
wherein said data from sensing changes in said property indicates active metabolic function in provided cells, and wherein changes in said property are monitored by at least one technique selected from the group consisting of proton spectroscopy, monitoring of C-13 labeled glucose, monitoring by P-31 MR spectroscopy, monitoring of local F-19 labeled metabolites, monitoring of Na-23 levels, and monitoring of 17O₂ gas conversion to H₂ 17O water.

18. The method of claim 1 wherein the sensing of a property within said region of a patient that is indicative of cell viability or nonviability of the provided colony of cells is used to quantitate the cell viability.

19. The method of claim 2 wherein the sensing of a property within said region of a patient that is indicative of cell viability or nonviability of the provided cells is used to quantitate the cell viability.

20. The method of claim 15 wherein the sensing of a property within said region of the organ that is indicative of cell viability or nonviability of the provided cells is used to quantitate the cell viability.
